(19) United States
(12) Patent Application Publication
(10) Pub. No.: US 2026/0092022 A1
(43) Pub. Date: Apr. 2, 2026

(54) HYDROCARBON PRODUCTION APPARATUS, HYDROCARBON PRODUCTION METHOD, AND NON-TRANSITORY COMPUTER READABLE MEDIUM

(71) Applicant: IHI Corporation, Tokyo (JP)

(72) Inventors: Daiki KAMIYA, Tokyo (JP); Takumi ENDO, Tokyo (JP)

(73) Assignee: IHI Corporation, Tokyo (JP)

(21) Appl. No.: 19/341,273

(22) Filed: Sep. 26, 2025

(30) Foreign Application Priority Data
Oct. 1, 2024 (JP) 2024-172019

(51) Int. Cl. C07C 1/12; G05B 15/02

(52) U.S. Cl. CPC C07C 1/12; G05B 15/02

(57) ABSTRACT

A hydrocarbon production apparatus includes: a reaction apparatus that causes a first raw gas including hydrogen and a second raw gas including carbon monoxide and/or carbon dioxide to react with each other, to thereby generate the hydrocarbon; a control device that executes first control of changing a flow rate of the first raw gas supplied to the reaction apparatus; a detection device that detects a concentration of the hydrocarbon included in a generated gas discharged from the reaction apparatus; and an acquisition device that acquires, for a plurality of times, the concentration of the hydrocarbon detected by the detection device in association with the flow rate of the first raw gas corresponding to the concentration of the hydrocarbon while the first control is being executed by the control device.

### BACKGROUND

### 1. Technical Field

The present disclosure relates to a hydrocarbon production apparatus, a hydrocarbon production method, and a non-transitory computer readable medium. The present application claims the benefit of priority based on Japanese Patent Application No. 2024-172019 filed on Oct. 1, 2024, the content of which is incorporated herein.

### 2. Description of the Related Art

There has been known a methane production apparatus which supplies carbon dioxide and hydrogen to a reactor, and produces methane in a reactor through a methanation reaction. In the methanation reaction, methane is most efficiently produced when a molar ratio of a supply amount of carbon dioxide to the reactor to a supply amount of hydrogen to the reactor is 1 to 4.

For example, in Patent Literature 1, there is disclosed a methane production apparatus including a first mass flow controller, a second mass flow controller, a gas analyzer, a reactor, and a control device. In a technology as disclosed in Patent Literature 1, the first mass flow controller supplies a mixed gas including carbon dioxide and hydrogen to the reactor. The second mass flow controller supplies hydrogen to the reactor. The gas analyzer detects a hydrogen/carbon dioxide ratio in the mixed gas to be led to the first mass flow controller. The control device refers to the hydrogen/carbon dioxide ratio detected by the gas analyzer, and outputs flow rate instruction amounts to the first mass flow controller and the second mass flow controller such that the hydrogen/carbon dioxide ratio of the mixed gas supplied to the reactor reaches 4.0.

### CITATION LIST

### Patent Literature

Patent Literature 1: JP 2021-116294 A

However, as in the technology as disclosed in Patent Literature 1, with a technology which controls the hydrogen/carbon dioxide ratio of the mixed gas supplied to the reactor based on the detection value of the gas analyzer which detects the hydrogen/carbon dioxide ratio on an upstream side of the reactor, there occurs a separation between an actual concentration of methane included in the generated gas generated in the reactor and a target concentration of methane for the generated gas. Specifically, with the technology as disclosed in Patent Literature 1, the actual flow rates of hydrogen and carbon dioxide supplied to the reactor deviate from the flow rate instruction amounts determined based on the target concentration by an amount corresponding to an error of the gas analyzer and errors of the mass flow controllers. Thus, there exists such a problem that the actual concentration of methane included in the generated gas separates from the target concentration.

### SUMMARY

The present disclosure has been made in view of the above-mentioned problem, and has an object to provide a hydrocarbon production apparatus, a hydrocarbon production method, and a non-transitory computer readable medium capable of appropriately acquiring supply flow rates of raw gases to a reaction apparatus for achieving a target concentration of hydrocarbon for a generated gas.

In order to solve the above-mentioned problem, according to one aspect of the present disclosure, there is provided a hydrocarbon production apparatus including: a reaction apparatus that causes a first raw gas including hydrogen and a second raw gas including carbon monoxide and/or carbon dioxide to react with each other, to thereby generate hydrocarbon; a control device that executes first control of changing one of a flow rate of the first raw gas supplied to the reaction apparatus or a flow rate of the second raw gas supplied to the reaction apparatus; a detection device that detects a concentration of the hydrocarbon included in a generated gas discharged from the reaction apparatus; and an acquisition device that acquires, for a plurality of times, the concentration of the hydrocarbon detected by the detection device in association with one of the flow rate of the first raw gas corresponding to the concentration of the hydrocarbon or the flow rate of the second raw gas corresponding to the concentration of the hydrocarbon while the first control is being executed by the control device.

The control device may execute second control of executing one of such control that the flow rate of the first raw gas reaches a flow rate of the first raw gas associated with a concentration closest to a target concentration out of the concentrations of the hydrocarbon acquired by the acquisition device or such control that the flow rate of the second raw gas reaches a flow rate of the second raw gas associated with the concentration closest to the target concentration out of the concentrations of the hydrocarbon acquired by the acquisition device.

In the first control, when the control device changes the flow rate in one of two directions, either to increase the flow rate or to reduce the flow rate, the control device may further change the flow rate in the one direction when the concentration of the hydrocarbon detected by the detection device increases; and the control device may change the flow rate the other direction when the concentration of the hydrocarbon detected by the detection device decreases.

The above-mentioned hydrocarbon production apparatus may further include a storage device that stores a plurality of set values indicating flow rates different from one another, and the control device may set the flow rate to any one set value out of the plurality of set values stored in the storage device in the first control.

The control device may change the flow rate in a stepwise manner in the first control.

The target concentration may be the maximum concentration out of the concentrations of the hydrocarbon acquired by the acquisition device.

In order to solve the above-mentioned problem, according to one aspect of the present disclosure, there is provided a hydrocarbon production method including: executing first control of changing one of a flow rate of a first raw gas supplied to a reaction apparatus or a flow rate of a second raw gas supplied to the reaction apparatus, the first raw gas including hydrogen, the second raw gas including carbon monoxide and/or carbon dioxide, the reaction apparatus causing the first raw gas and the second raw gas to react with each other, to thereby generate hydrocarbon; detecting a concentration of the hydrocarbon included in a generated gas discharged from the reaction apparatus; and acquiring, for a plurality of times, the detected concentration of the hydrocarbon in association with one of the flow rate of the first raw gas corresponding to the concentration of the hydrocarbon or the flow rate of the second raw gas corresponding to the concentration of the hydrocarbon while the first control is being executed.

In order to solve the above-mentioned problem, according to one aspect of the present disclosure, there is provided a non-transitory computer readable medium storing a program for causing a computer to function as: a control device that executes first control of changing one of a flow rate of a first raw gas supplied to a reaction apparatus or a flow rate of a second raw gas supplied to the reaction apparatus, the first raw gas including hydrogen, the second raw gas including carbon monoxide and/or carbon dioxide, the reaction apparatus causing the first raw gas and the second raw gas to react with each other, to thereby generate hydrocarbon; and an acquisition device that acquires, for a plurality of times, a detected concentration of the hydrocarbon in association with one of the flow rate of the first raw gas corresponding to the concentration of the hydrocarbon or the flow rate of the second raw gas corresponding to the concentration of the hydrocarbon while the first control is being executed by the control device.

According to the present disclosure, it is possible to appropriately acquire the supply flow rates of the raw gases to the reaction apparatus for achieving the target concentration of the hydrocarbon for the generated gas.

### BRIEF DESCRIPTION OF THE DRAWINGS

FIG. 1 is a schematic diagram of a hydrocarbon production apparatus according to at least one embodiment of the present disclosure.

FIG. 2 is a block diagram for illustrating an example of a functional configuration of a control apparatus in the at least one embodiment.

FIG. 3 is a flowchart for illustrating an example of a flow of processing of a hydrocarbon production method according to the at least one embodiment.

FIG. 4 is a graph for showing an example of a flow rate change in a first raw gas and a concentration change in methane in first control in the at least one embodiment.

FIG. 5 is a graph for showing an example of the flow rate change in the first raw gas and the concentration change in methane in the first control in a modification example.

### DESCRIPTION OF THE EMBODIMENTS

Now, with reference to the attached drawings, at least one embodiment of the present disclosure is described in detail. The dimensions, materials, and other specific numerical values represented in the at least one embodiment are merely examples used for facilitating the understanding of the disclosure, and do not limit the present disclosure unless otherwise particularly noted. Elements having substantially the same functions and configurations herein and in the drawings are denoted by the same reference symbols to omit redundant description thereof. Further, illustration of elements with no direct relationship to the present disclosure is omitted.

### 1. Overview of Hydrocarbon Production Apparatus

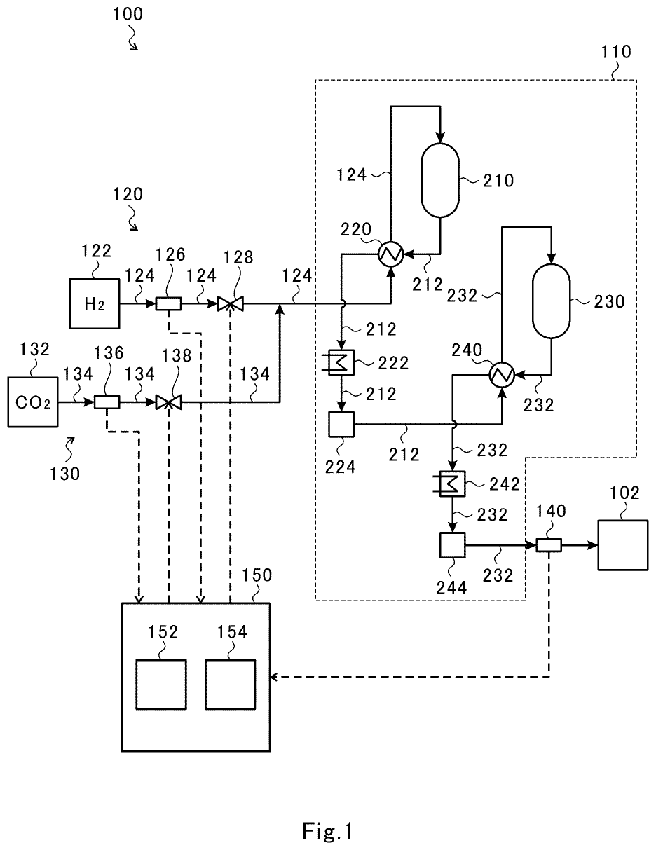

First, with reference to FIG. 1, description is given of an overview of a hydrocarbon production apparatus 100 according to the at least one embodiment of the present disclosure. FIG. 1 is a schematic diagram of the hydrocarbon production apparatus 100 according to the at least one embodiment of the present disclosure.

As illustrated in FIG. 1, the hydrocarbon production apparatus 100 according to the at least one embodiment includes a reaction apparatus 110, a first supply device 120, a second supply device 130, a detection device 140, and a control apparatus 150. In FIG. 1, the arrows in the solid lines indicate flows of gasses and liquids. Moreover, in FIG. 1, the arrows in the broken lines indicate flows of signals.

The reaction apparatus 110 causes a first raw gas including hydrogen and a second raw gas including carbon monoxide and/or carbon dioxide to react with each other, to thereby generate hydrocarbon. In the at least one embodiment, there is exemplified a case in which the first raw gas includes hydrogen and inevitable impurities and the second raw gas includes carbon dioxide and inevitable impurities. In this case, in the reaction apparatus 110, a synthesis reaction between hydrogen and carbon dioxide takes place, resulting in the production of the hydrocarbon. The synthesis reaction between hydrogen and carbon dioxide is an exothermic reaction. For example, the synthesis reaction between hydrogen and carbon dioxide is a reaction given by formulae (1) to (4).

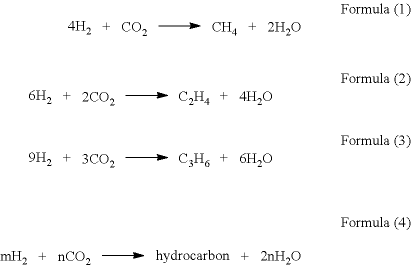

Methane is produced through the reaction given by the formula (1). Ethylene (olefine) is produced through the reaction given by the formula (2). Propylene (olefine) is produced through the reaction given by the formula (3). Hydrocarbon is produced through the Fischer-Tropsch (FT) synthesis reaction given by the formula (4). In the at least one embodiment, there is exemplified a case in which the reaction given by the formula (1) takes place in the reaction apparatus 110, to thereby produce methane.

It suffices that the first raw gas include at least hydrogen. Moreover, it suffices that the second raw gas include carbon monoxide and/or carbon dioxide.

Moreover, in the at least one embodiment, the reaction apparatus 110 includes, for example, a first reactor 210, a first discharge tube 212, a first heat exchanger 220, a first condenser 222, a first gas-liquid separator 224, a second reactor 230, a second discharge tube 232, a second heat exchanger 240, a second condenser 242, and a second gas-liquid separator 244.

To the first reactor 210, the first raw gas is supplied from the first supply device 120 described later, and the second raw gas is supplied from the second supply device 130. In the first reactor 210, a catalyst which promotes the reaction between the first raw gas and the second raw gas is accommodated. The catalyst is, for example, a catalyst which promotes a reaction given by the formula (1). The first reactor 210 is maintained at a temperature at which the reaction given by the formula (1) is efficiently executed by a heat medium (not shown).

The first discharge tube 212 is a tube which causes the first reactor 210 and the second reactor 230 to communicate with each other. That is, the first reactor 210 and the second reactor 230 are serially connected to each other via the first discharge tube 212. To the first discharge tube 212, the first heat exchanger 220, the first condenser 222, and the first gas-liquid separator 224 are provided in the stated order.

The first heat exchanger 220 executes heat exchange between a first generated gas flowing through the first discharge tube 212 and a mixed gas flowing through a first supply tube 124 described later. The first generated gas includes hydrogen, carbon dioxide, methane, and vapor. The mixed gas is a mixed gas of the first raw gas and the second raw gas. In the at least one embodiment, the first heat exchanger 220 transfers heat of the first generated gas to the mixed gas.

The first condenser 222 cools the first generated gas, to thereby condense the vapor included in the first generated gas. The first gas-liquid separator 224 separates water (liquid) generated by the first condenser 222 from the first generated gas.

To the second reactor 230, the first generated gas from which the water has been removed is supplied via the first discharge tube 212. In the second reactor 230, a catalyst which promotes the reaction between the first raw gas and the second raw gas is accommodated. The catalyst is, for example, a catalyst which promotes a reaction given by the formula (1). The second reactor 230 is maintained at a temperature at which the reaction given by the formula (1) is efficiently executed by a heat medium (not shown).

The second discharge tube 232 is a tube which causes the second reactor 230 and a supply destination 102 to communicate with each other. When the hydrocarbon production apparatus 100 produces methane as the hydrocarbon, the supply destination 102 is, for example, a combustion device which uses methane as a fuel, a boiler including a combustion device which uses methane as a fuel, or an electric power generator including a combustion device which uses methane as a fuel. To the second discharge tube 232, the second heat exchanger 240, the second condenser 242, and the second gas-liquid separator 244 are provided in the stated order.

The second heat exchanger 240 executes heat exchange between a second generated gas flowing through the second discharge tube 232 and the first generated gas flowing through the first discharge tube 212. The second generated gas includes hydrogen, carbon dioxide, methane, and vapor. A concentration of methane included in the second generated gas is higher than a concentration of methane included in the first generated gas. Concentrations of hydrogen and carbon dioxide included in the second generated gas are lower than concentrations of hydrogen and carbon dioxide included in the first generated gas, respectively. In the at least one embodiment, the second heat exchanger 240 transfers heat of the second generated gas to the first generated gas.

The second condenser 242 cools the second generated gas, to thereby condense the vapor included in the second generated gas. The second gas-liquid separator 244 separates water (liquid) generated by the second condenser 242 from the second generated gas.

The first supply device 120 supplies the first raw gas to the reaction apparatus 110. In the at least one embodiment, the first supply device 120 supplies the first raw gas to the first reactor 210. The first supply device 120 includes, for example, a supply source 122 for the first raw gas, the first supply tube 124, a first flowmeter 126, and a first flow rate adjustment valve 128.

The supply source 122 for the first raw gas is, for example, a hydrogen cylinder or a water electrolysis device and a blower.

The first supply tube 124 is a pipe which connects the supply source 122 for the first raw gas and the first reactor 210 to each other. To the first supply tube 124, the first flowmeter 126, the first flow rate adjustment valve 128, and the first heat exchanger 220 are provided in the stated order.

The first flowmeter 126 measures a flow rate of the first raw gas flowing through the first supply tube 124. The first flow rate adjustment valve 128 adjusts the flow rate of the first raw gas flowing through the first supply tube 124. The first flow rate adjustment valve 128 adjusts an opening degree of a flow path formed in the first supply tube 124.

The second supply device 130 supplies the second raw gas to the reaction apparatus 110. In the at least one embodiment, the second supply device 130 supplies the second raw gas to the first reactor 210. The second supply device 130 includes, for example, a supply source 132 for the second raw gas, a second supply tube 134, a second flowmeter 136, and a second flow rate adjustment valve 138.

The supply source 132 for the second raw gas is, for example, a carbon dioxide cylinder or a recovering device which recovers carbon dioxide from a combustion exhaust gas and the like and a blower.

The second supply tube 134 is a tube which connects the supply source 132 for the second raw gas and the first supply tube 124 to each other. In the at least one embodiment, the second supply device 130 supplies the second raw gas to the first reactor 210 via the second supply tube 134 and the first supply tube 124. In detail, the second supply tube 134 connects the supply source 132 for the second raw gas and a portion between the first flow rate adjustment valve 128 and the first heat exchanger 220 in the first supply tube 124. To the second supply tube 134, the second flowmeter 136 and the second flow rate adjustment valve 138 are provided in the stated order.

The second flowmeter 136 measures a flow rate of the second raw gas flowing through the second supply tube 134. The second flow rate adjustment valve 138 adjusts the flow rate of the second raw gas flowing through the second supply tube 134. The second flow rate adjustment valve 138 adjusts an opening degree of a flow path formed in the second supply tube 134.

The detection device 140 detects a concentration of the hydrocarbon included in the generated gas discharged from the reaction apparatus 110. In the at least one embodiment, the detection device 140 detects the concentration of methane included in the second generated gas flowing through the second discharge tube 232 between the second gas-liquid separator 244 and the supply destination 102. As the detection device 140, for example, a product name “smart gas detector SD-1 series” manufactured by Riken Keiki Co., Ltd. can be used.

The control apparatus 150 includes one or a plurality of processors 152 and one or a plurality of memories 154 connected to the processor 152. The processor 152 includes, for example, a central processing unit (CPU). The memory 154 includes, for example, a read only memory (ROM) and a random access memory (RAM). The ROM is a storage element which stores programs, operation parameters, and the like used by the CPU. The RAM is a storage element which temporarily stores data such as variables and parameters used for processing executed by the CPU.

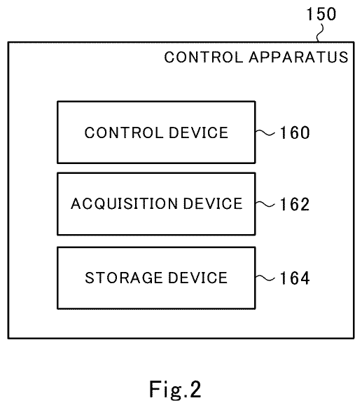

FIG. 2 is a block diagram for illustrating an example of a functional configuration of the control apparatus 150 in the at least one embodiment. For example, as illustrated in FIG. 2 the control apparatus 150 also functions as a control device 160, an acquisition device 162, and a storage device 164.

Various types of processing including processing executed by one or a plurality of devices including the control device 160, the acquisition device 162, and the storage device 164 and described later can be executed by the processor 152. In detail, the various types of processing are executed by the processor 152 executing the programs stored in the memory 154. A function of the storage device 164 is implemented by the memory 154. However, the functions of the control apparatus 150 may be distributed to a plurality of apparatus, and a plurality of functions may be implemented by one apparatus.

The control device 160 executes first control. The first control is control of changing the flow rate of the first raw gas supplied to the reaction apparatus 110. In the at least one embodiment, the control device 160 controls the first supply device 120, to thereby execute the first control. In detail, the control device 160 acquires a measurement value of the first flowmeter 126 of the first supply device 120, and adjusts the opening degree of the first flow rate adjustment valve 128 of the first supply device 120 such that the measurement value of the first flowmeter 126 reaches a flow rate instruction value. In the at least one embodiment, the control device 160 does not change the flow rate of the second raw gas supplied to the reaction apparatus 110 during execution of the first control, the second control, and a normal operation. For example, the control device 160 fixes the flow rate of the second raw gas supplied to the reaction apparatus 110 to a flow rate of the generated gas required in the supply destination 102 during the execution of the first control, the second control, and the normal operation.

The control device 160, for example, changes the flow rate in a stepwise manner in the first control. Specifically, the control device 160 holds the flow rate of the first raw gas at a first predetermined flow rate for a predetermined hold time, and changes the flow rate of the first raw gas from the first predetermined flow rate to a second predetermined flow rate when the hold time has elapsed in the first control. After that, when the flow rate of the first raw gas is changed to the second predetermined flow rate, the control device 160 holds the flow rate of the first raw gas at the second predetermined flow rate for a hold time. The hold time is set such that the hold time is equal to or longer than a time from the supply of the mixed gas (first raw gas and second raw gas) to the first reactor 210 of the reaction apparatus 110 to a full reaction between the first raw gas and the second raw gas. For example, the hold time is set such that the hold time is equal to or longer than a time from when the mixed gas is supplied to the first reactor 210 of the reaction apparatus 110 to when the generated gas reaches the detection device 140. The hold time for holding at the first predetermined flow rate and the hold time for holding at the second predetermined flow rate may be equal to each other or different from each other.

Moreover, the control device 160 may execute the second control after the first control is executed. The second control is control of controlling the flow rate of the first raw gas such that a flow rate of the first raw gas associated with a concentration closest to a target concentration out of concentrations of the hydrocarbon acquired by the acquisition device 162 described later is reached. In the at least one embodiment, the control device 160 controls the first supply device 120 to execute the second control. The flow rate of the first raw gas associated with the concentration closest to the target concentration is hereinafter sometimes referred to as “first flow rate.”

Moreover, the target concentration may be the maximum concentration out of the concentrations of the hydrocarbon acquired by the acquisition device 162.

The acquisition device 162 acquires, for a plurality of times, the concentration of the hydrocarbon detected by the detection device 140 in association with the flow rate of the first raw gas corresponding to the concentration of the hydrocarbon while the first control is being executed by the control device 160. In detail, the acquisition device 162 successively acquires the detection result obtained by the detection device 140 while the flow rate of the first raw gas is being changed by the control device 160. Moreover, the acquisition device 162 associates the concentration of methane detected by the detection device 140 and the flow rate of the first raw gas corresponding to this concentration of methane with each other. For example, the acquisition device 162 acquires the detection result obtained by the detection device 140 while the flow rate is maintained at the first predetermined flow rate, and acquires the detection result obtained by the detection device 140 while the flow rate is maintained at the second predetermined flow rate. After that, the acquisition device 162 stores, in storage device 164 in association with the first predetermined flow rate, the detection result acquired while the flow rate is maintained at the first predetermined flow rate. Moreover, the acquisition device 162 stores, in storage device 164 in association with the second predetermined flow rate, the detection result acquired while the flow rate is maintained at the second predetermined flow rate.

The storage device 164 stores, for example, information on the concentration of the hydrocarbon acquired by the acquisition device 162 and the flow rate of the first raw gas corresponding to this concentration of the hydrocarbon associated with each other.

### 2. Hydrocarbon Production Method

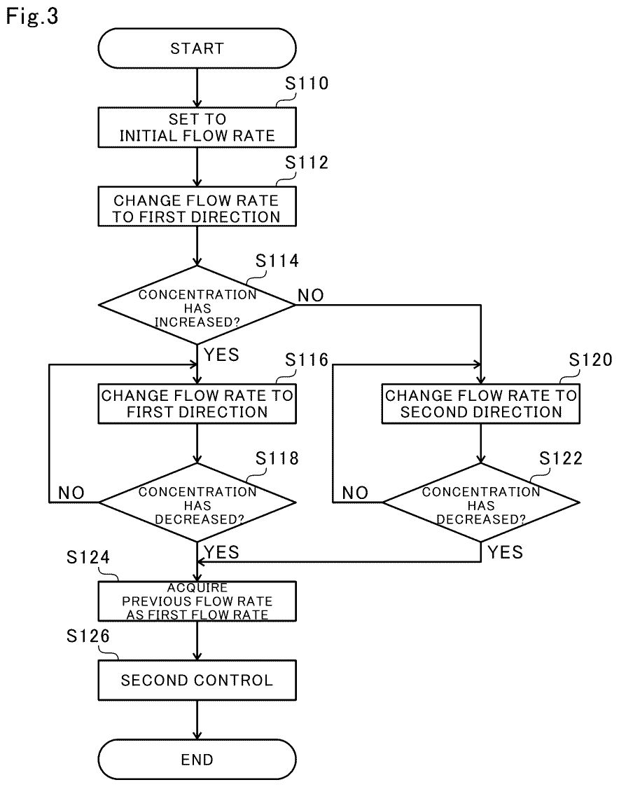

Next, with reference to FIG. 3, description is now given of a flow of a hydrocarbon production method according to the at least one embodiment of the present disclosure. FIG. 3 is a flowchart for illustrating an example of the flow of processing of the hydrocarbon production method according to the at least one embodiment.

As illustrated in FIG. 3, the hydrocarbon production method according to the at least one embodiment includes initial flow rate setting processing S110, first flow rate change processing S112, increase determination processing S114, second flow rate change processing S116, decrease determination processing S118, third flow rate change processing S120, decrease determination processing S122, first flow rate acquisition processing S124, and second control processing S126. The processing from the initial flow rate setting processing S110 to the first flow rate acquisition processing S124 corresponds to the above-mentioned first control.

### [Initial Flow Rate Setting Processing S110]

The control device 160 controls the first supply device 120 to set the flow rate of the first raw gas to a first initial flow rate. Moreover, the control device 160 controls the second supply device 130 to set the flow rate of the second raw gas to a second initial flow rate. The first initial flow rate may be, for example, a flow rate within a range from 3.80 times or more to 4.21 times or less of the flow rate of the generated gas required in the supply destination 102. The second initial flow rate may be, for example, a flow rate 1.00 time of the flow rate of the generated gas required in the supply destination 102. The control device 160 does not change the flow rate of the second raw gas from the second initial flow rate in the first control, the second control, and the normal operation. That is, the control device 160 fixes the flow rate of the second raw gas supplied by the second supply device 130 in the first control, the second control, and the normal operation.

Moreover, when the hold time has elapsed since the flow rate of the first raw gas was set to the first initial flow rate by the control device 160, the acquisition device 162 acquires the concentration of methane detected by the detection device 140. As described above, the hold time is set so as to be equal to or longer than the time from the supply of the mixed gas (first raw gas and second raw gas) to the first reactor 210 of the reaction apparatus 110 to the full reaction between the first raw gas and the second raw gas. For example, the holding time is a time equal to or longer than a time from when the mixed gas is supplied to the first reactor 210 of the reaction apparatus 110 to when the generated gas reaches the detection device 140. Moreover, the acquisition device 162 stores, in storage device 164 in association with the first initial flow rate, the concentration of methane acquired in this initial flow rate setting processing S110.

### [First Flow Rate Change Processing S112]

The control device 160 changes, for example, the flow rate of the first raw gas in one of two directions, either to increase the flow rate or to reduce the flow rate thereof. The direction set in the first flow rate change processing S112 is hereinafter sometimes referred to as “first direction.” Moreover, another direction being an opposite direction of the one direction set in the first flow rate change processing S112 is sometimes referred to as “second direction.” Moreover, in the at least one embodiment, the control device 160 increases or reduces the flow rate of the first raw gas by a first change amount. The first change amount may be, for example, 0.1% or more and 5% or less of the flow rate of the second raw gas determined based on the target flow rate of the generated gas required in the supply destination 102.

When a hold time has elapsed since a time at which the control device 160 changed the flow rate of the first raw gas to the first direction, the acquisition device 162 acquires the concentration of methane detected by the detection device 140. Moreover, the acquisition device 162 stores, in the storage device 164, the flow rate of the first raw gas set in this first flow rate change processing S112 and the concentration of methane acquired in this first flow rate change processing S112 in association with each other.

### [Increase Determination Processing S114]

The control device 160 determines whether or not the concentration of methane acquired in the first flow rate change processing S112 has increased from the concentration of methane acquired in the initial flow rate setting processing S110. As a result, when the control device 160 determines that the concentration has increased (YES in Step S114), the control device 160 advances the process to the second flow rate change processing S116. Meanwhile, when the control device 160 determines that the concentration has not increased, that is, has decreased (NO in Step S114), the control device 160 advances the process to the third flow rate change processing S120.

### [Second Flow Rate Change Processing S116]

The control device 160 changes the flow rate of the first raw gas toward the first direction. In the at least one embodiment, the control device 160 increases or reduces the flow rate of the first raw gas by the first change amount also in the second flow rate change processing S116.

Moreover, when a hold time has elapsed since a time at which the control device 160 changed the flow rate of the first raw gas to the first direction, the acquisition device 162 acquires the concentration of methane detected by the detection device 140. Moreover, the acquisition device 162 stores, in the storage device 164, the flow rate of the first raw gas set in this second flow rate change processing S116 and the concentration of methane acquired in this second flow rate change processing S116 in association with each other.

### [Decrease Determination Processing S118]

The control device 160 determines whether or not the concentration of methane acquired in the second flow rate change processing S116 executed for this time has decreased from the concentration of methane acquired in the second flow rate change processing S116 executed for the previous time. When the second flow rate change processing S116 has been executed only once, the control device 160 determines whether or not the concentration of methane acquired in the second flow rate change processing S116 executed for this time has decreased from the concentration of methane acquired in the first flow rate change processing S112. As a result, when the control device 160 determines that the concentration has decreased (YES in Step S118), the control device 160 advances the process to the first flow rate acquisition processing S124. Meanwhile, when the control device 160 determines that the concentration has not decreased, that is, has increased (NO in Step S118), the control device 160 repeats the processing from the second flow rate change processing S116.

### [Third Flow Rate Change Processing S120]

The control device 160 changes the flow rate of the first raw gas toward the second direction. In the at least one embodiment, the control device 160 increases or reduces the flow rate of the first raw gas by the first change amount also in the third flow rate change processing S120.

Moreover, when a hold time has elapsed since a time at which the control device 160 changed the flow rate of the first raw gas to the second direction, the acquisition device 162 acquires the concentration of methane detected by the detection device 140. Moreover, the acquisition device 162 stores, in the storage device 164, the flow rate of the first raw gas set in this third flow rate change processing S120 and the concentration of methane acquired in this third flow rate change processing S120 in association with each other.

### [Decrease Determination Processing S122]

The control device 160 determines whether or not the concentration of methane acquired in the third flow rate change processing S120 executed for this time has decreased from the concentration of methane acquired in the third flow rate change processing S120 executed for the previous time. When the third flow rate change processing S120 has been executed only once, the control device 160 determines whether or not the concentration of methane acquired in the third flow rate change processing S120 executed for this time has decreased from the concentration of methane acquired in the first flow rate change processing S112. As a result, when the control device 160 determines that the concentration has decreased (YES in Step S122), the control device 160 advances the process to the first flow rate acquisition processing S124. Meanwhile, when the control device 160 determines that the concentration has not decreased, that is, has increased (NO in Step S122), the control device 160 repeats the processing from the third flow rate change processing S120.

### [First Flow Rate Acquisition Processing S124]

The acquisition device 162 acquires the flow rate of the first raw gas set immediately before the decrease in concentration of methane as the first flow rate associated with a concentration closest to the maximum concentration (target concentration).

### [Second Control Processing S126]

The control device 160 controls the first supply device 120 such that the flow rate reaches the first flow rate acquired in the first flow rate acquisition processing S124, and transitions to the normal operation.

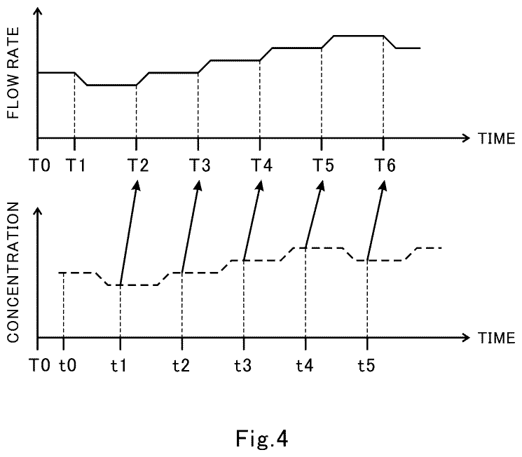

FIG. 4 is a graph for showing an example of the flow rate change in the first raw gas and the concentration change in methane in the first control in the at least one embodiment. An upper graph of FIG. 4 shows the flow rate change in first raw gas in the first control. A lower graph of FIG. 4 shows the concentration change in methane in the first control.

As illustrated in FIG. 4, the control device 160 sets the flow rate of the first raw gas to the first initial flow rate at, for example, a time T0, and the acquisition device 162 acquires the concentration of methane at a time to after the time T0 (Step S110).

After that, the control device 160 changes the flow rate toward the direction for reducing the flow rate of the first raw gas at a time T1 after the time to, and the acquisition device 162 acquires the concentration of methane at a time t1 after the time T1 (Step S112).

Here, as shown in FIG. 4, for example, when the concentration of methane acquired at the time t1 has decreased from the concentration of methane acquired at the time to (NO in Step S114), the control device 160 changes the flow rate toward the direction for increasing the flow rate of the first raw gas at a time T2 after the time t1, and the acquisition device 162 acquires the concentration of methane at a time t2 after the time T2 (Step S120).

Then, for example, when the concentration of methane acquired at the time t2 has increased from the concentration of methane acquired at the time t1 (NO in Step S122), the control device 160 changes the flow rate toward the direction for increasing the flow rate of the first raw gas at a time T3 after the time t2, and the acquisition device 162 acquires the concentration of methane at a time t3 after the time T3 (Step S120).

Further, for example, when the concentration of methane acquired at the time t3 has increased from the concentration of methane acquired at the time t2 (NO in Step S122), the control device 160 changes the flow rate toward the direction for increasing the flow rate of the first raw gas at a time T4 after the time t3, and the acquisition device 162 acquires the concentration of methane at a time t4 after the time T4 (Step S120).

Still further, for example, when the concentration of methane acquired at the time t4 has increased from the concentration of methane acquired at the time t3 (NO in Step S122), the control device 160 changes the flow rate toward the direction for increasing the flow rate of the first raw gas at a time T5 after the time t4, and the acquisition device 162 acquires the concentration of methane at a time t5 after the time T5 (Step S120).

After that, for example, when the concentration of methane acquired at the time t5 has decreased from the concentration of methane acquired at the time t4 (YES in Step S122), the acquisition device 162 acquires, as the first flow rate, the flow rate of the first raw gas set at the time T4 immediately before T5 (Step S124). After that, the control device 160 controls the first supply device 120 at a time T6 after the time t5 such that the first flow rate is reached, and transitions to the normal operation (Step S126).

### 3. Program

Moreover, there is provided a program for causing a computer to function as: a control device 160 that executes first control of changing one of a flow rate of a first raw gas supplied to a reaction apparatus 110 or a flow rate of a second raw gas supplied to the reaction apparatus 110, the first raw gas including hydrogen, the second raw gas including carbon monoxide and/or carbon dioxide, the reaction apparatus 110 causing the first raw gas and the second raw gas to react with each other, to thereby generate hydrocarbon; and an acquisition device 162 that acquires, for a plurality of times, a detected concentration of the hydrocarbon in association with one of the flow rate of the first raw gas corresponding to the concentration of the hydrocarbon or the flow rate of the second raw gas corresponding to the concentration of the hydrocarbon while the first control is being executed by the control device 160. The above program may be stored in a non-transitory computer-readable storage medium and provided as a storage medium.

### 4. Conclusion

As described above, the hydrocarbon production apparatus according to the at least one embodiment includes: a reaction apparatus 110 that causes a first raw gas including hydrogen and a second raw gas including carbon monoxide and/or carbon dioxide to react with each other, to thereby generate hydrocarbon; a control device 160 that executes first control of changing one of a flow rate of the first raw gas supplied to the reaction apparatus 110 or a flow rate of the second raw gas supplied to the reaction apparatus 110; a detection device 140 that detects a concentration of the hydrocarbon included in a generated gas discharged from the reaction apparatus 110; and an acquisition device 162 that acquires, for a plurality of times, the concentration of the hydrocarbon detected by the detection device 140 in association with one of the flow rate of the first raw gas corresponding to the concentration of the hydrocarbon or the flow rate of the second raw gas corresponding to the concentration of the hydrocarbon while the first control is being executed by the control device 160.

For example, when the flow rate of the first raw gas supplied to the reaction apparatus 110 is controlled based on the first flowmeter 126 included in the first supply device 120, the actual flow rate of the first raw gas supplied to the reaction apparatus 110 deviates from the flow rate instruction value by an amount corresponding to an error of the first flowmeter 126 and an error of the first flow rate adjustment valve 128. Thus, even when the flow rate instruction value is determined based on the target concentration, the actual concentration of the hydrocarbon included in the generated gas separates from the target concentration. Thus, the control device 160 in the at least one embodiment executes the first control of changing the flow rate of the first raw gas supplied to the reaction apparatus 110, and the acquisition device 162 acquires, for the plurality of times, the concentration of the hydrocarbon detected by the detection device 140 in association with the flow rate of the first raw gas corresponding to the concentration of the hydrocarbon while the first control is being executed. As a result, the hydrocarbon production apparatus 100 according to the at least one embodiment can appropriately acquire the supply flow rate of the first raw gas to the reaction apparatus 110 for achieving the target concentration of the hydrocarbon for the generated gas.

Moreover, the control device 160 in the at least one embodiment changes the flow rate of the first raw gas higher (for example, may be 3.80 times or more and 4.21 times or less) in supply flow rate than the second raw gas as the first control. Thus, the hydrocarbon production apparatus 100 according to the at least one embodiment can more easily adjust the flow rate in the first control.

The control device 160 may execute second control of executing one of such control that the flow rate of the first raw gas reaches a flow rate of the first raw gas associated with a concentration closest to a target concentration out of the concentrations of the hydrocarbon acquired by the acquisition device 162 or such control that the flow rate of the second raw gas reaches a flow rate of the second raw gas associated with the concentration closest to the target concentration out of the concentrations of the hydrocarbon acquired by the acquisition device 162.

As a result, the hydrocarbon production apparatus 100 according to the at least one embodiment can cause the concentration of the hydrocarbon included in the generated gas to stably approach the target concentration.

In the first control, when the control device changes the flow rate in one of two directions, either to increase the flow rate or to reduce the flow rate, the control device 160 may further change the flow rate in the one direction when the concentration of the hydrocarbon detected by the detection device 140 increases; and the control device 160 may change the flow rate the other direction when the concentration of the hydrocarbon detected by the detection device 140 decreases.

As a result, the hydrocarbon production apparatus 100 according to the at least one embodiment can appropriately acquire the first flow rate regardless of whether the first initial flow rate is higher or lower than the first flow rate. Moreover, the hydrocarbon production apparatus 100 according to the at least one embodiment can appropriately acquire the first flow rate without consideration of disturbance of the atmospheric temperature, the atmospheric pressure, and the like.

The control device 160 may change the flow rate in the stepwise manner in the first control.

As a result, the hydrocarbon production apparatus 100 according to the at least one embodiment can associate the detected concentration of the hydrocarbon and the flow rate of the first raw gas corresponding to the concentration of the hydrocarbon in additional consideration of a delay which is caused by a reaction time in the reaction apparatus 110 and corresponds to a time until the change in flow rate of the first raw gas is reflected to the change in concentration of the hydrocarbon.

The target concentration may be the maximum concentration out of the concentrations of the hydrocarbon acquired by the acquisition device 162.

As a result, the hydrocarbon production apparatus 100 according to the at least one embodiment can maximize production efficiently of the hydrocarbon.

Moreover, as described above, the hydrocarbon production method according to the at least one embodiment includes: executing first control of changing one of a flow rate of a first raw gas supplied to a reaction apparatus 110 or a flow rate of a second raw gas supplied to the reaction apparatus 110, the first raw gas including hydrogen, the second raw gas including carbon monoxide and/or carbon dioxide, the reaction apparatus 110 causing the first raw gas and the second raw gas to react with each other, to thereby generate hydrocarbon; detecting a concentration of the hydrocarbon included in a generated gas discharged from the reaction apparatus 110; and acquiring, for a plurality of times, the detected concentration of the hydrocarbon in association with one of the flow rate of the first raw gas corresponding to the concentration of the hydrocarbon or the flow rate of the second raw gas corresponding to the concentration of the hydrocarbon while the first control is being executed.

As a result, the hydrocarbon production method according to the at least one embodiment can appropriately acquire the supply flow rate of the first raw gas to the reaction apparatus 110 for achieving the target concentration of the hydrocarbon for the generated gas.

Further, as described above, a program according to the at least one embodiment causes a computer to function as: a control device 160 that executes first control of changing one of a flow rate of a first raw gas supplied to a reaction apparatus 110 or a flow rate of a second raw gas supplied to the reaction apparatus 110, the first raw gas including hydrogen, the second raw gas including carbon monoxide and/or carbon dioxide, the reaction apparatus 110 causing the first raw gas and the second raw gas to react with each other, to thereby generate hydrocarbon; and an acquisition device 162 that acquires, for a plurality of times, a detected concentration of the hydrocarbon in association with one of the flow rate of the first raw gas corresponding to the concentration of the hydrocarbon or the flow rate of the second raw gas corresponding to the concentration of the hydrocarbon while the first control is being executed by the control device 160.

As a result, the program according to the at least one embodiment can appropriately acquire the supply flow rate of the first raw gas to the reaction apparatus 110 for achieving the target concentration of the hydrocarbon for the generated gas.

### 5. Modification Example

In the above-mentioned embodiment, there is exemplified the case in which the control device 160 changes the flow rate of the first raw gas based on the acquired increase and decrease in concentration of methane in the first control. However, the control device 160 may control the flow rate of the first raw gas based on a set value set in advance regardless of an increase or a decrease in acquired concentration of methane.

In a modification example, the storage device 164 stores, for example, a plurality of the set values indicating flow rates different from one another. Moreover, the control device 160 sets the flow rate of the first raw gas to any one set value out of the plurality of set values stored in the storage device 164 in the first control. Also in the modification example, the control device 160 does not change the flow rate of the second raw gas from the second initial flow rate in the first control, the second control, and the normal operation. That is, the control device 160 fixes the flow rate of the second raw gas supplied by the second supply device 130 in the first control, the second control, and the normal operation.

Moreover, a difference between two set values close in value may be, for example, 0.1% or more and 5% or less of the flow rate of the second raw gas determined based on the target flow rate of the generated gas required in the supply destination 102.

After that, the acquisition device 162 stores, in the storage device 164, the set value and the acquired concentration of methane in association with each other. As a result, the hydrocarbon production apparatus 100 according to the modification example can appropriately acquire the supply flow rate of the first raw gas to the reaction apparatus 110 for achieving the target concentration of the hydrocarbon for the generated gas. After that, the acquisition device 162 acquires, as the first flow rate, the set value associated with the methane concentration closest to the concentration of methane of the target concentration out of the concentrations of methane stored in the storage device 164.

In the modification example, a first set value set for the first time by the control device 160 may be the flow rate of the first raw gas used for the normal operation for the previous time.

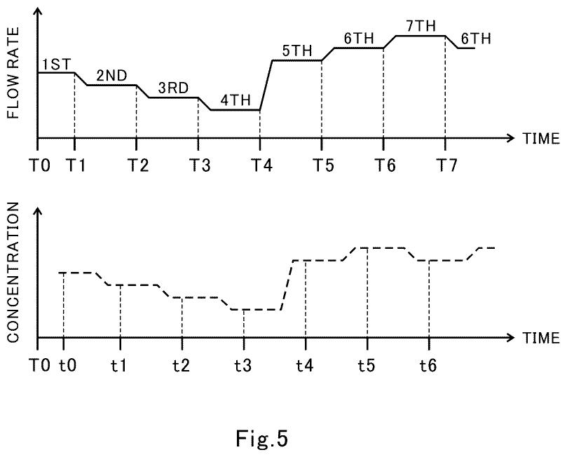

FIG. 5 is a graph for showing an example of the flow rate change in first raw gas and the concentration change in methane in the first control in the modification example. An upper graph of FIG. 5 shows the flow rate change in first raw gas in the first control in the modification example. A lower graph of FIG. 5 shows the concentration change in methane in the first control in the modification example.

As illustrated in FIG. 5, the control device 160 sets the flow rate of the first raw gas to the first set value at, for example, the time T0, and the acquisition device 162 acquires the concentration of methane at the time to after the time T0. After that, the acquisition device 162 stores, in storage device 164 in association with the first set value, the concentration of methane acquired at the time to.

After that, the control device 160 changes the flow rate of the first raw gas from the first set value to a second set value at the time T1 after the time to, and the acquisition device 162 acquires the concentration of methane at the time t1 after the time T1. After that, the acquisition device 162 stores, in storage device 164 in association with the second set value, the concentration of methane acquired at the time t1. The second set value indicates, for example, a flow rate lower than the first set value.

Moreover, the control device 160 changes the flow rate of the first raw gas from the second set value to a third set value at the time T2 after the time t1, and the acquisition device 162 acquires the concentration of methane at the time t2 after the time T2. After that, the acquisition device 162 stores, in storage device 164 in association with the third set value, the concentration of methane acquired at the time t2. The third set value indicates, for example, a flow rate lower than the second set value.

After that, the control device 160 changes the flow rate of the first raw gas from the third set value to a fourth set value at the time T3 after the time t2, and the acquisition device 162 acquires the concentration of methane at the time t3 after the time T3. After that, the acquisition device 162 stores, in storage device 164 in association with the fourth set value, the concentration of methane acquired at the time t3. The fourth set value indicates, for example, a flow rate lower than the third set value.

Moreover, the control device 160 changes the flow rate of the first raw gas from the fourth set value to a fifth set value at the time T4 after the time t3, and the acquisition device 162 acquires the concentration of methane at the time t4 after the time T4. After that, the acquisition device 162 stores, in storage device 164 in association with the fifth set value, the concentration of methane acquired at the time t4. The fifth set value indicates, for example, a flow rate larger than the first set value.

After that, the control device 160 changes the flow rate of the first raw gas from the fifth set value to a sixth set value at the time T5 after the time t4, and the acquisition device 162 acquires the concentration of methane at the time t5 after the time T5. After that, the acquisition device 162 stores, in storage device 164 in association with the sixth set value, the concentration of methane acquired at the time t5. The sixth set value indicates, for example, a flow rate larger than the fifth set value.

Moreover, the control device 160 changes the flow rate of the first raw gas from the sixth set value to a seventh set value at the time T6 after the time t5, and the acquisition device 162 acquires the concentration of methane at the time t6 after the time T6. After that, the acquisition device 162 stores, in storage device 164 in association with the seventh set value, the concentration of methane acquired at the time t6. The seventh set value indicates, for example, a flow rate larger than the sixth set value.

After that, when the control device 160 finishes the setting for all of the set values stored in the storage device 164, the acquisition device 162 acquires, as the first flow rate, the set value of the flow rate of the first raw gas associated with a concentration closest to the target concentration out of the concentrations of methane stored in the storage device 164. For example, in the example shown in FIG. 5, the acquisition device 162 acquires a sixth set value as the first flow rate. After that, the control device 160 controls the first supply device 120 such that the first flow rate is reached at, for example, a time T7 after the time t6, and transitions to the normal operation.

The at least one embodiment has been described above with reference to the attached drawings, but, needless to say, the present disclosure is not limited to the at least one embodiment. It is apparent that those skilled in the art may arrive at various alternations and modifications within the appended claims, and those examples are construed as naturally falling within the technical scope of the present disclosure.

For example, in the at least one embodiment, there is exemplified a case in which the second raw gas is formed of carbon dioxide and the inevitable impurities. However, the second raw gas may be formed of carbon monoxide and/or carbon dioxide and the inevitable impurities. When hydrogen and carbon monoxide are supplied to the reaction apparatus 110, in the reaction apparatus 110, a synthesis reaction between hydrogen and carbon monoxide takes place, resulting in the production of the hydrocarbon. The synthesis reaction between hydrogen and carbon monoxide is an exothermic reaction. For example, the synthesis reaction between hydrogen and carbon monoxide is a reaction given by formulae (5) to (8).

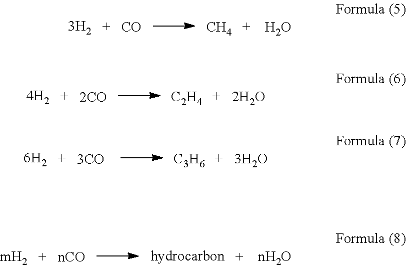

Moreover, in the at least one embodiment, the first control executed by the control device 160 is the control of changing the flow rate of the first raw gas supplied to the reaction apparatus 110. However, the first control executed by the control device 160 may be control of changing the flow rate of the second raw gas supplied to the reaction apparatus 110. In this case, for example, the control device 160 controls the second supply device 130, to thereby execute the first control. In detail, the control device 160 acquires a measurement value of the second flowmeter 136 of the second supply device 130, and adjusts the opening degree of the second flow rate adjustment valve 138 of the second supply device 130 such that the measurement value of the second flowmeter 136 reaches the flow rate instruction value. In this case, in the first control, the control device 160 increases or reduces the flow rate of the second raw gas by the first change amount. The first change amount at this time may be, for example, 0.1% or more and 5% or less of the flow rate of the first raw gas determined based on the target flow rate of the generated gas required in the supply destination 102. Moreover, in this case, the control device 160 does not change the flow rate of the first raw gas supplied to the reaction apparatus 110 during the execution of the first control, the second control, and the normal operation. For example, the control device 160 fixes the flow rate of the first raw gas supplied to the reaction apparatus 110 to a range from 3.80 times or more to 4.21 times or less of the flow rate of the generated gas required in the supply destination 102 during the execution of the first control, the second control, and the normal operation. Moreover, the acquisition device 162 acquires the concentration of the hydrocarbon detected by the detection device 140 in association with the flow rate of the second raw gas corresponding to the concentration of the hydrocarbon for a plurality of times during the execution of the first control. After that, the control device 160 executes the second control of controlling the flow rate of the second raw gas such that the flow rate reaches a flow rate (second flow rate) of the second raw gas associated with a concentration closest to the target concentration out of the concentrations of the hydrocarbon acquired by the acquisition device 162. For example, the control device 160 controls the second supply device 130, to thereby execute the second control. As a result, the hydrocarbon production apparatus 100 can appropriately acquire the supply flow rate of the second raw gas to the reaction apparatus 110 for achieving the target concentration of the hydrocarbon for the generated gas. Moreover, the hydrocarbon production apparatus 100 can cause the concentration of the hydrocarbon included in the generated gas to stably approach the target concentration.

Moreover, in the at least one embodiment, there is exemplified the case in which the control device 160 changes the flow rate in the stepwise manner in the first control. However, the control device 160 may continuously change the flow rate in the first control.

Moreover, in the at least one embodiment, there is exemplified the case in which the target concentration is the maximum concentration out of the concentrations of the hydrocarbon acquired by the acquisition device 162. However, the target concentration may be determined based on a calorific value required in the supply destination 102. Moreover, the target concentration may be determined based on a plurality of ratios among materials including hydrogen, carbon monoxide, carbon dioxide, and the hydrocarbon required in the supply destination 102.

The present disclosure can contribute to, for example, Goal 7 “Ensure access to affordable, reliable, sustainable and modern energy for all” and Goal 13 “Take urgent action to combat climate change and its impacts” in Sustainable Development Goals (SDGs).

What is claimed is:

1. A hydrocarbon production apparatus, comprising:
a reaction apparatus that causes a first raw gas including hydrogen and a second raw gas including carbon monoxide and/or carbon dioxide to react with each other, to thereby generate hydrocarbon;
a control device that executes first control of changing one of a flow rate of the first raw gas supplied to the reaction apparatus or a flow rate of the second raw gas supplied to the reaction apparatus;
a detection device that detects a concentration of the hydrocarbon included in a generated gas discharged from the reaction apparatus; and
an acquisition device that acquires, for a plurality of times, the concentration of the hydrocarbon detected by the detection device in association with one of the flow rate of the first raw gas corresponding to the concentration of the hydrocarbon or the flow rate of the second raw gas corresponding to the concentration of the hydrocarbon while the first control is being executed by the control device.

2. The hydrocarbon production apparatus according to claim 1, wherein the control device executes second control of executing one of such control that the flow rate of the first raw gas reaches a flow rate of the first raw gas associated with a concentration closest to a target concentration out of the concentrations of the hydrocarbon acquired by the acquisition device or such control that the flow rate of the second raw gas reaches a flow rate of the second raw gas associated with the concentration closest to the target concentration out of the concentrations of the hydrocarbon acquired by the acquisition device.

3. The hydrocarbon production apparatus according to claim 1, wherein, in the first control, when the control device changes the flow rate in one of two directions, either to increase the flow rate or to reduce the flow rate,
the control device further changes the flow rate in the one direction when the concentration of the hydrocarbon detected by the detection device increases; and
the control device changes the flow rate the other direction when the concentration of the hydrocarbon detected by the detection device decreases.

4. The hydrocarbon production apparatus according to claim 1, further comprising a storage device that stores a plurality of set values indicating flow rates different from one another,
wherein the control device sets the flow rate to any one set value out of the plurality of set values stored in the storage device in the first control.

5. The hydrocarbon production apparatus according to claim 1, wherein the control device changes the flow rate in a stepwise manner in the first control.

6. The hydrocarbon production apparatus according to claim 2, wherein the target concentration is the maximum concentration out of the concentrations of the hydrocarbon acquired by the acquisition device.

7. A hydrocarbon production method, comprising:
executing first control of changing one of a flow rate of a first raw gas supplied to a reaction apparatus or a flow rate of a second raw gas supplied to the reaction apparatus, the first raw gas including hydrogen, the second raw gas including carbon monoxide and/or carbon dioxide, the reaction apparatus causing the first raw gas and the second raw gas to react with each other, to thereby generate hydrocarbon;
detecting a concentration of the hydrocarbon included in a generated gas discharged from the reaction apparatus; and
acquiring, for a plurality of times, the detected concentration of the hydrocarbon in association with one of the flow rate of the first raw gas corresponding to the concentration of the hydrocarbon or the flow rate of the second raw gas corresponding to the concentration of the hydrocarbon while the first control is being executed.

8. A non-transitory computer readable medium storing a program for causing a computer to function as:
a control device that executes first control of changing one of a flow rate of a first raw gas supplied to a reaction apparatus or a flow rate of a second raw gas supplied to the reaction apparatus, the first raw gas including hydrogen, the second raw gas including carbon monoxide and/or carbon dioxide, the reaction apparatus causing the first raw gas and the second raw gas to react with each other, to thereby generate hydrocarbon; and
an acquisition device that acquires, for a plurality of times, a detected concentration of the hydrocarbon in association with one of the flow rate of the first raw gas corresponding to the concentration of the hydrocarbon or the flow rate of the second raw gas corresponding to the concentration of the hydrocarbon while the first control is being executed by the control device.
